(19) United States
(12) Patent Application Publication
(10) Pub. No.: US 2002/0002373 A1
(43) Pub. Date: Jan. 3, 2002

(54) Arrangement for eliminating, or at least for reducing the size of, interspaces between three elements arranged one behind the other in the longitudinal direction

(76) Inventors: Raimar Boehlke, Raubling (DE); Werner Schwarz, Ruhpolding (DE); Thomas Ischinger, Muenchen (DE); Thomas Ehrl, Essen (DE)

Correspondence Address: McLane, Graf, Raulerson & Middleton, Professional Association, 900 Elm Street, P.O. Box 326, Manchester, NH, 03105-0326, US

(21) Appl. No.: 09/847,676

(22) Filed: May 2, 2001

(30) Foreign Application Priority Data
Sep. 8, 1999 (DE) PCT/DE00/03118
Jan. 31, 2001 (DE) 201 01 699.0

(51) Int. Cl. A61B 17/12

(52) U.S. Cl. 606/139

(57) ABSTRACT

For guiding at least two sutures through a wall of a membrane, of a balloon or of a surface, in particular of an artery of an individual, in the vicinity of the edge region of an opening provided therein and for drawing back out of the abovementioned opening the suture ends guided through the relevant wall, the invention provides a shaft-like suture-guide device which, in its longitudinal direction, contains a rear suture-feed part (3), a front suture-accommodating part (4) and a central suture-release/suture-clamping part located therebetween; the central suture-release/ suture-clamping part (5) can be expanded in one axial direction such that that membrane, balloon or surface or vessel wall of an artery of an individual which is located in the region of the relevant central suture-release/suture-clamping part (5) is thus subjected to tensioning, on account of which those edge regions of the abovementioned wall which are provided transversely to said tensioning direction draw together toward one another.

The invention relates to an arrangement for guiding at least two sutures through a wall of a membrane, of a balloon or of a surface, in particular of an artery of an individual, in the vicinity of the edge region of an opening provided therein, and formed if appropriate by cutting in and/or cutting out, and for drawing back out of the abovementioned opening the suture ends guided through the relevant edge region, having a shaft-like suture-guide device in which the sutures fastened on needles are guided in guide and/or accommodating openings such that, by means of the relevant suture-guide device, they can be guided through the abovementioned wall, in the vicinity of the edge region of the relevant opening, and drawn back out of the abovementioned opening again such that, by virtue of the suture ends being drawn together, and if appropriate knotted, outside the relevant opening, the latter can be closed.

An arrangement of the abovementioned type is used mainly in cases in which there is provided in the wall of a membrane, of a balloon or of a surface an opening which is to be closed and for the closure of which access from both sides of the relevant wall is not possible. This is the case, in particular, when the wall containing the opening which is to be closed belongs to an artery of an individual. In these cases, it is only possible for access to the opening which is to be closed in each case to be gained from the outside.

WO 94/08516 has already disclosed a suturing device of the type mentioned in the introduction in which up to four needles connected to sutures can be guided, by means of a pushing device, through a wall of a blood vessel in the vicinity of the edge region of an opening located therein and accommodated by an accommodating device located within the relevant blood vessel in the region of the abovementioned opening. This accommodating device is formed by an intercepting-cage-like needle-accommodating device which first of all is guided through the relevant opening in the collapsed state and then is widened in the relevant opening, in order thereafter to butt against the edge region of the relevant opening within the blood vessel. The needles guided through the edge region of the abovementioned opening can be accommodated by said intercepting-cage-like needle-accommodating device, which is then rotated about its longitudinal axis in order to secure the relevant needles. Thereafter, the relevant intercepting-cage-like needle-accommodating device, with the needles contained in it, is collapsed in order to be drawn out in its entirety through the abovementioned opening. During this operation, the sutures connected to the needle ends are drawn out of supply magazines, drawn through the insertion locations of the abovementioned needles, in the vicinity of the edge region of the abovementioned opening, and drawn out of the relevant opening again. Outside the abovementioned opening, the suture ends may then be drawn together, and if appropriate knotted, in order to close the relevant opening.

Although the known arrangement considered above is, in principle, of relatively straightforward construction, problems may nevertheless arise in the case of the needles guided through in the vicinity of the edge region of the respective opening being accommodated and secured in the intercepting-cage-like needle-accommodating device if one or more needles cannot be secured by said needle-accommodating device. In such cases, complicated intervention is then necessary in order for the needles contained in the blood vessel to be removed again.

Also known, from WO 94/13211, is an arrangement which is intended for guiding two sutures through a wall of a blood vessel in the vicinity of the edge region of an opening formed therein and contains a needle carrier, which is to be introduced into the relevant opening, and a needle-accommodating device, which is located outside the relevant opening. The needle carrier is provided with sutures, of which the ends are connected to needles. During use, first of all, the abovementioned needle carrier is introduced in its entirety through the abovementioned opening into the blood vessel which is to be closed, and then the needles are guided through the edge region of the relevant opening from the inside to the outside. In this case, the rest of the needle-carrier parts initially still remain in the blood vessel. Thereafter, these needle-carrier parts are drawn out of the blood vessel through the abovementioned opening, with the result that merely the suture connected to the needles which have already been guided out remains in the relevant blood vessel. By virtue of the needles being drawn back further, the relevant suture, finally, is tensioned in the interior of the relevant blood vessel, with the result that the suture ends can then be knotted. The relevant opening is then consequently closed.

The known arrangement being considered here does indeed allow, in principle, sutures to be guided through the wall of a blood vessel in the vicinity of the edge region of an opening provided therein; however, it is also the case here that the reliability in conjunction with the needle ends being accommodated in the needle-accommodating device is at least critical. This is because, if one or other of the needles is not accommodated reliably by the needle-accommodating device, additional intervention is also necessary in this case in order to avoid complications. For dimensioning reasons, the access to the artery has to be widened here. The relevant known arrangement is thus not minimally invasive.

Also known, from WO 95/13021, is an arrangement for guiding at least one suture through the wall of a blood vessel of an individual in the vicinity of the edge region of an opening provided therein. This known arrangement has a shaft-like suture-guide device, at the tip of which there is provided a nose piece which has a needle-deflecting path and is connected to the shaft-like suture-guide device via a region of reduced cross section. Provided in this shaft-like suture-guide device is a needle-feed opening which is aligned in relation to the inlet side of the needle-deflecting path in the nose piece. Also provided in the shaft-like suture-guide device is a second needle-guide opening, which is aligned in relation to the outlet side of the needle-deflecting path in the nose piece. On account of this construction, the known arrangement being considered here functions as follows. First of all the entire arrangement is introduced into that opening of the blood vessel of an individual which is to be closed to such an extent that the wall of the relevant opening butts against the abovementioned region of reduced cross section, via which the nose piece is connected to the shaft-like suture-guide device. A needle connected to a suture is then moved forwards through the needle-feed opening of the shaft-like suture-guide device in the direction of the nose piece, the relevant needle here piercing the wall of the blood vessel in the vicinity of the edge region of the abovementioned opening and then being deflected in the deflecting path of the nose piece such that it then pierces the edge region of the relevant vessel wall from the inside to the outside. Thereafter, the relevant needle is guided back through the abovementioned further needle-guide opening of the shaft-like suture-guide device again, with the result that the edge region of the abovementioned opening thus has a suture passing through it at two diametrically opposite locations. This suture then has to be guided out of the deflecting path via a suture-release slot arrangement connected to said path, with the result that, thereafter, the entire arrangement can be drawn out of the opening of the abovementioned blood vessel.

The known arrangement considered above does indeed, in principle, allow at least one suture to be guided through the wall of blood vessel, in the vicinity of the edge region of an opening provided therein, at two diametrically opposite locations; however, the abovementioned needle-deflecting design occasionally poses problems in practice since, as a result of the relatively pronounced curvature of the deflecting path provided in the abovementioned nose piece, it is only possible to use flexible needles or small needles, which meanwhile can cause problems in terms of guiding such needles through vessel walls.

Finally, U.S. Pat. No. 5,860,990 has also already disclosed a suturing arrangement for guiding the ends of a suture through the wall of a blood vessel of an individual in the vicinity of the edge region of an opening provided therein. In this known arrangement, a suture supply with loop-like suture ends is introduced, by means of a shaft-like suture-feed device, through the relevant opening into the blood vessel. Provided outside the relevant shaft-like suture-feed device, at two diametrically opposite locations, are needle-like suture-accommodating means which, following piercing of the vessel wall in the vicinity of the edge region of the relevant opening, are to accommodate the loop-like suture ends and then to draw them outwards out of the blood vessel.

Although the last-considered known arrangement allows, in principle, loop-like ends of a suture to be guided through a blood-vessel wall in the vicinity of the edge region of an opening provided therein, this design is nevertheless also problematic to use in terms of the abovementioned loop-like suture ends being accommodated. This is because reliable accommodation of the relevant loop-like suture ends is only ensured when the relevant suture ends are secured by the shaft-like suture-feed device in a defined position in which the needle-like suture-accommodating means can also grip these loop-like suture ends. In practice, this can only be achieved from time to time with considerable difficulty.

Accordingly, the object of the invention is to find a way in which, in the case of an arrangement of the type mentioned in the introduction, the at least two sutures provided can be guided through the wall of a membrane, of a balloon or of a surface, in particular of an artery of an individual, in the vicinity of the edge region of an opening provided therein in a relatively straightforward but nevertheless reliable manner.

The above object is achieved according to the invention, in the case of an arrangement of the type mentioned in the introduction, in that the suture-guide device, in its longitudinal direction, has a rear suture-feed part, a front suture-accommodating part and a central suture-release/suture-clamping part located therebetween, and in that the abovementioned central suture-release/suture-clamping part can be rotated at least relative to the front suture-accommodating part and has such a cross section that, in at least one rotary position, it allows the sutures fed from the rear suture-feed part to be introduced into accommodating openings exposed in the front suture-accommodating part and, in a rotary position differing from the abovementioned rotary position, it allows the sutures accommodated in the relevant accommodating openings together with the needles to be secured for drawing the entire suture-guide device out of the abovementioned opening.

The invention has the advantage that, with relatively low outlay, it ensures that at least two sutures are guided reliably through the wall of a membrane, of a balloon or of a surface, in particular of an artery of an individual, in the vicinity of the edge region of an opening provided therein and that the sutures guided through in the vicinity of the abovementioned edge region are drawn back out of the relevant opening. For effective use of the arrangement according to the invention, the relevant opening may, if appropriate, be cut out for the introduction of said arrangement. In this case, the present invention utilizes a relatively straightforward design principle according to which merely the central suture-release/suture-clamping part need be rotatable relative to the other arrangement parts in order in one rotary position, a suture-release position, to allow the sutures to be guided through in the vicinity of the edge region of the abovementioned opening and in another rotary position, a suture-clamping position, for the needles accommodated with the sutures in the front suture-accommodating part to be clamped firmly such that the entire arrangement can be drawn out of the relevant opening. In this case, the sutures are drawn along through the abovementioned wall in the vicinity of the edge region of the abovementioned opening in order then to be drawn together, and if appropriate knotted, outside said opening. A knot pusher known per se may then be used for this operation, which will not be described in any more detail here. Surgical sutures are suitable for use as sutures for the case where the arrangement according to the invention is an arrangement for closing arteries or blood vessels in general. Eversion seams may then consequently be produced. The advantage of minimally invasive closure is achieved in the case of using the present invention for closing an artery.

Expedient developments of the invention are included in the subclaims.

The rear suture-feed part, that is to say that provided at the proximal end of the arrangement, the central suture-release/suture-clamping part and the front suture-accommodating part, that is to say that provided at the distal end of the arrangement, each expediently have an oval-shaped cross section. This allows optimal functioning of the relevant arrangement, which is beneficial, in particular, in the case where the abovementioned opening is located in an artery wall of an individual, human or animal. This artery wall may then be positioned in its entirety against the oval-shaped cross section of the relevant arrangement parts. The central suture-release/suture-clamping part serves in this case, as will become clear hereinbelow, as a wound-edge tensioner in the artery-wall opening which is to be closed.

It is sufficient here on occasion if the rear suture-feed part, the central suture-release/suture-clamping part and the front suture-accommodating part of the suture-guide device each have the same oval-shaped cross section at least in their adjacent regions. This advantageously allows the entire arrangement to be easily introduced into the respective opening and guided out of the same, in the vicinity of the border region of which at least two sutures are to be guided through the wall containing the relevant opening.

It is particularly advantageous, furthermore, if the cross section of the central suture-release/suture-clamping part has a smaller thickness than the cross sections of the rear suture-feed part and of the front suture-accommodating part. As a result, the relevant suture-release/suture-clamping part may be positioned against the edge of the abovementioned opening, in the vicinity of the edge region of which at least two sutures are to be guided through the wall containing the relevant opening, so that the penetration locations for guiding the relevant sutures through are located as far away as possible from the edge of the abovementioned opening. This is quite considerably advantageous for the closure of an opening provided in an artery wall.

It is also particularly advantageous if the rear suture-feed part, the central suture-release/suture-clamping part and the front suture-accommodating part of the suture-guide device can all be rotated relative to one another. This rotatability of the individual arrangement parts relative to one another allows very flexible functioning, which is beneficial, in particular, in the cases where more than two sutures are to be guided through the wall of a membrane, of a balloon or of a surface, in particular of an artery of an individual, in the vicinity of the edge region of an opening provided in said wall.

A particularly straightforward arrangement design is achieved when the central suture-release/suture-clamping part is formed by a region of reduced cross section of the rear suture-feed part. This advantageously makes it possible to manage with just two arrangement parts which can be rotated relative to one another.

In the case of this arrangement design being considered here, the region of reduced cross section of the rear suture-feed part may expediently be formed by a separate part, which is connected to the rear suture-feed part. This makes it possible to produce the relevant arrangement parts relatively easily.

The central suture-release/suture-clamping part preferably has a thickness which corresponds to the thickness of the wall of a membrane, of a balloon or of a surface, in particular of an artery of an individual. This makes it optimally possible for at least two sutures to be guided through the abovementioned wall in the vicinity of the edge region of an opening provided in said wall.

Expediently provided in the rear suture-feed part and in the front suture-accommodating part are m groups, where m≧2 in each case, of n longitudinal and accommodating holes located one beside the other, where n≧1. This measure has the advantage that a relatively large number n of sutures in groups m can be guided through the wall of a membrane, of a balloon or of a surface, in particular of an artery of an individual, in the vicinity of the border region of an opening provided in said wall.

In a particularly straightforward arrangement design, the guide openings of the rear suture-feed part are formed by longitudinal holes in which the sutures fastened on the needles can be displaced by means of separate pushers and can be introduced, via the central suture-release/suture-clamping part, into elongate accommodating holes aligned with the longitudinal holes and forming the abovementioned accommodating openings, said accommodating holes belonging to the front accommodating part located in its one position mentioned above. The relevant pushers advantageously allow the abovementioned needles to be reliably and easily guided through the wall of a membrane, of a balloon or of a surface in the vicinity of the edge region of the opening provided in the relevant wall.

In this case, the accommodating holes of the front suture-accommodating part preferably have such a depth that at least the needles located at the front ends of the sutures can be accommodated in their entirety in said accommodating holes. In this case, the relevant accommodating holes may preferably be configured such that they only allow the needles, once introduced, to be drawn out with difficulty, if at all.

In order for the needles to be secured reliably in the abovementioned accommodating holes, it is possible for said accommodating holes in the front suture-accommodating part expediently to have their respective longitudinal axis running at an angle in relation to the longitudinal axis of the longitudinal holes of the rear suture-feed part. This measure allows the needles to be clamped firmly in a relatively straightforward and reliable manner in the relevant accommodating holes.

In order for it to be possible for the sutures to be guided through as far away as possible from the edge region of the opening provided in the abovementioned wall, the abovementioned longitudinal holes are preferably located at the edge of the suture-feed part and are covered by a sheeting part secured on said suture-feed part. This has the advantage of a particularly low level of design outlay for the desired determination of the longitudinal holes.

In this case, the longitudinal holes in the rear suture-feed part are expediently positioned in the direction of the outer circumference thereof to such an extent that part of the outer circumference of the needles is located outside the outer circumference of the rear suture-feed part. This measure also helps to position the lead-through openings for the abovementioned sutures as far away as possible from the edge of the abovementioned opening.

Preferably located in the suture-feed part, alongside the longitudinal holes, are supply chambers in which there are accommodated additional needles which are connected to further sutures and, once the needles initially provided in the abovementioned longitudinal holes have been introduced into the accommodating holes provided in the front suture-accommodating part and the pushers advanced for said introduction operation have subsequently been drawn back into a withdrawal position, in which the relevant supply chambers are released, pass into the abovementioned longitudinal holes, in which they can be introduced, by means of the abovementioned pushers, into accommodating holes provided in the front suture-accommodating part. This measure is advantageously used when more than two sutures are to be guided through the wall of a membrane, of a balloon or of a surface, in particular of an artery of an individual, in the vicinity of the edge region of an opening provided in said wall.

For accommodating the abovementioned further needles, use is expediently made of the same accommodating holes in which the needles which were initially located in the longitudinal holes are accommodated. For this purpose, the relevant accommodating holes may each have such a length that they allow two needles to be accommodated one behind the other. It is also possible, however, for the relevant accommodating holes to be configured such that they are capable of accommodating in each case two needles firmly one beside the other.

In order for it to be possible for the abovementioned further needles to be guided out of the abovementioned supply chambers in a straightforward manner, a spring force is utilized. This may be produced, for example, by compression springs or helical springs.

If further accommodating holes are provided in the front suture-accommodating part, before the further needles accommodated in the abovementioned supply chambers are introduced into the abovementioned further accommodating holes of the front suture-accommodating part, the latter is rotated relative to the rear suture-feed part such that the further accommodating holes provided in the front suture-accommodating part are then aligned in relation to the longitudinal holes provided in the rear suture-feed part. It is thus possible, for example, for four sutures to be guided through a wall at equal intervals from one another around the edge region of the abovementioned opening.

Before the abovementioned further needles are introduced into the accommodating holes of the front suture-accommodating part, the rear suture-feed part and the front suture-accommodating part are expediently rotated relative to the central suture-release/suture-clamping part. This gives the advantage that, for example, four sutures are guided through a wall at equal intervals from one another around the edge region of the abovementioned opening.

The central suture-release/suture-clamping part is expediently connected to a hand grip by means of a sleeve which passes through the rear suture-feed part in a rotatable manner, and the rear suture-feed part and the front suture-accommodating part are expediently connected to rotary adjustment wheels, if appropriate, via a sleeve arrangement arranged coaxially with the abovementioned sleeve. This has the advantage of a relatively straightforward design for the adjustment and movement of the individual arrangement parts.

The abovementioned rotary adjustment wheels are expediently connected to latching catches which allow the relevant rotary adjustment wheels, and thus the rear suture-feed part and front suture-accommodating part connected thereto, to be adjusted into determined angle positions relative to the hand grip, and thus to the central suture-release/suture-clamping part. This gives the advantage of an adjustment device, for the individual arrangement parts, which can be adjusted particularly straightforwardly, but nevertheless effectively.

The rotary adjustment wheels for the rear suture-feed part and for the front suture-accommodating part are expediently coupled to a locking/release mechanism such that the displacement of the needles by the respective pusher is released only with the suture-feed part and suture-accommodating part aligned in relation to one another. This has the advantage that reliable displacement of the needles by the respective pusher can only take place when the suture-feed part and the suture-accommodating part are aligned in relation to one another, which is quite particularly beneficial from the point of view of preventing any risks or accidents. It is only in this relative position of the suture-feed part and of the suture-accommodating part that it is ensured that the needles cannot be guided out of the arrangement unintentionally or incorrectly.

It is advantageous here for the displacement of the needles to be released in the case where the central suture-release/suture-clamping part is located in its suture-release position. This measure ensures that, with the suture-feed part and suture-accommodating part aligned in relation to one another, the abovementioned needles can only be displaced when the suture-release/suture-clamping part located therebetween is located in its suture-release position, that is to say in the position which is actually provided for guiding the needles with the sutures connected thereto through a wall of a membrane, of a balloon or of a surface, in particular of an artery of an individual.

The rear suture-feed part and the front suture-accommodating part preferably have a guide element passing through them, said guide element serving for introducing the entire arrangement into the abovementioned opening. It is expediently possible here, with the aid of the relevant guide element, for the front suture-accommodating part to be rotated relative to the rear suture-feed part. This has the advantage that the relevant guide element, in addition to its guide function, can also be utilized as a rotary element. In this case, the abovementioned guide element may preferably be formed by a guide wire.

In the case of the arrangement construction being considered here, the rear suture-feed part expediently has a sleeve part, which encloses the guide element, passing through it, it being possible for the central suture-release/suture-clamping part to be rotated relative to the rear suture-feed part by means of said sleeve part. In the case of the arrangement design being considered here, this allows relatively straightforward rotatability of the arrangement parts considered.

Finally, on its surface directed towards the central suture-release/suture-clamping part, it is also possible for the front suture-accommodating part to have swing-open and swing-in spring elements which, with rotation of the front suture-accommodating part from its starting position relative to the central suture-release/suture-clamping part, swing open in a first direction such that the surface of the relevant front suture-accommodating part is correspondingly increased in size in the direction of the central suture-release/suture-clamping part, and which, with guidance of the suture-accommodating part back into its abovementioned starting position, can be moved back into their swung-in state again. This gives the advantage that, in a relatively straightforward manner, there is an increase in size of the surface by which the front suture-accommodating part butts against the inside of the wall through the opening of which the front suture-accommodating part is guided, which is associated with greater reliability during use of the arrangement according to the invention.

The invention is explained in more detail hereinbelow, by way of example, with reference to drawings in which the same designations are used in each case for the same or mutually corresponding parts and/or elements.

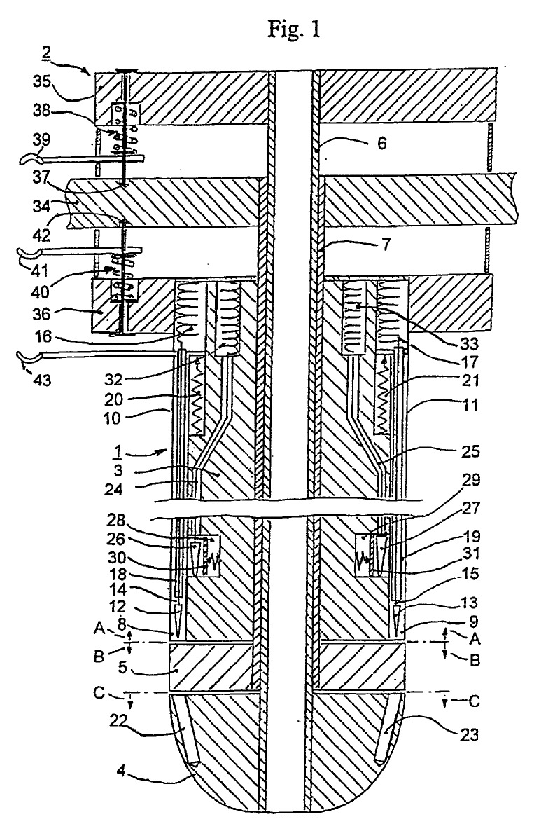

FIG. 1 shows a sectional view of a first embodiment of the invention of an arrangement embodying the invention.

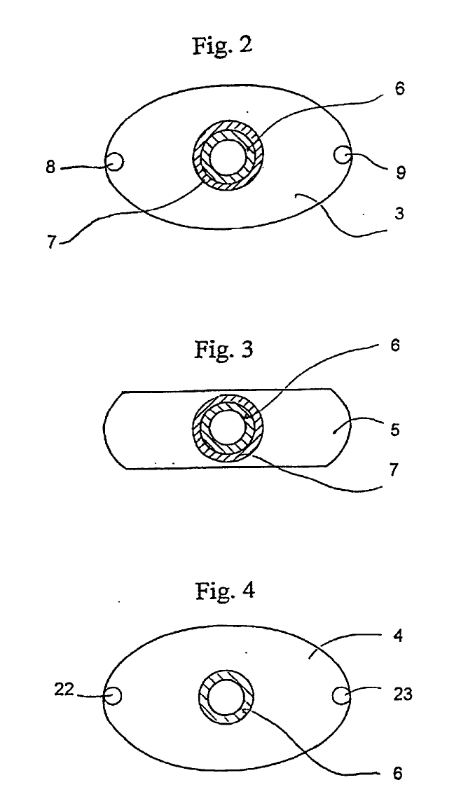

FIG. 2 shows a plan view of a rear suture-feed part of the arrangement illustrated in FIG. 1 in accordance with the section line A-A indicated therein.

FIG. 3 shows a plan view of a central suture-release/suture-clamping part of the arrangement illustrated in FIG. 1 in accordance with the section line B-B indicated therein.

FIG. 4 shows a plan view of a front suture-accommodating part of the arrangement illustrated in FIG. 1 in accordance with the section line C-C indicated therein.

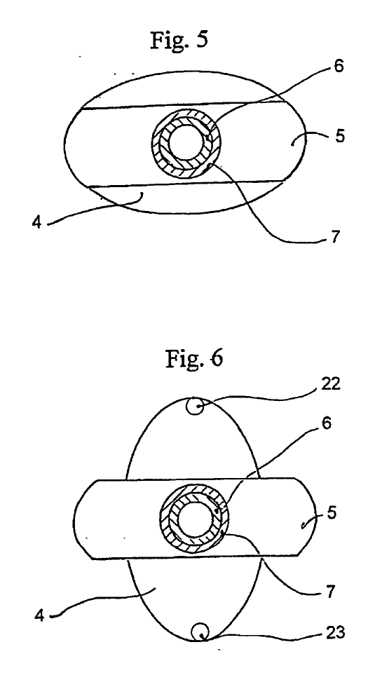

FIG. 5 shows a plan view of the central suture-release/suture-clamping part and the front suture-accommodating part of the arrangement according to FIG. 1 in a starting position.

FIG. 6 shows the arrangement parts illustrated in FIG. 5 in a rotary position differing from the rotary position therein.

FIG. 7 shows the arrangement parts illustrated in FIG. 5 in another rotary position.

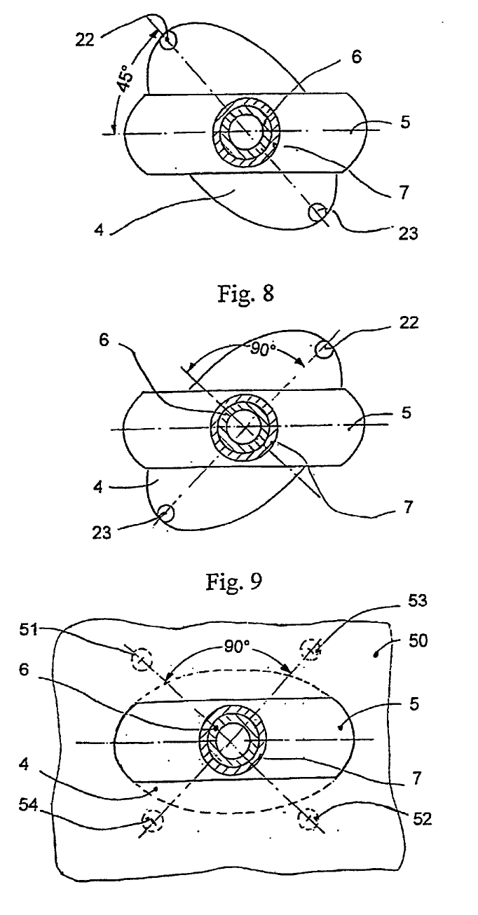

FIG. 8 shows the arrangement parts illustrated in FIG. 5 in yet a further rotary position.

FIG. 9 shows the arrangement parts illustrated in FIG. 5 once again in their starting position, once needles with sutures have previously been guided through holes in the vicinity of the edge region of an opening provided in an indicated wall in accordance with the rotary positions of the arrangement parts according to FIGS. 7 and 8.

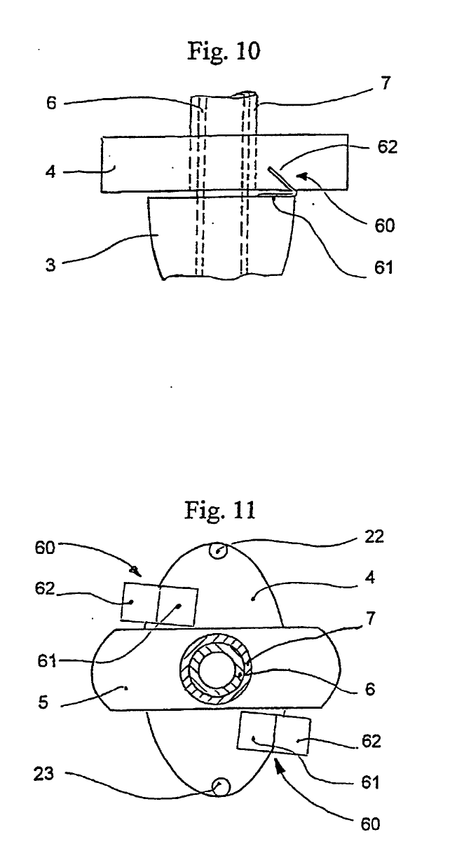

FIG. 10 shows a side view which illustrates that the front suture-accommodating part is provided with swing-open and swing-in spring elements, of which only one can be seen in FIG. 10.

FIG. 11 shows a plan view of the arrangement parts illustrated in FIG. 10 with the two spring elements swung open.

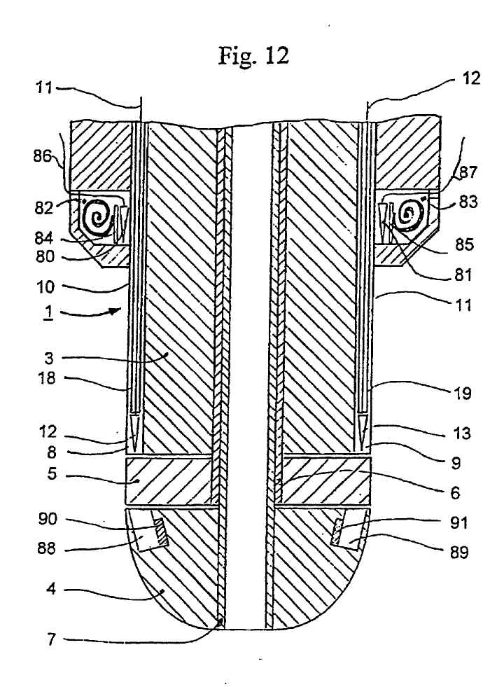

FIG. 12 shows a modification of the arrangement illustrated in FIG. 1.

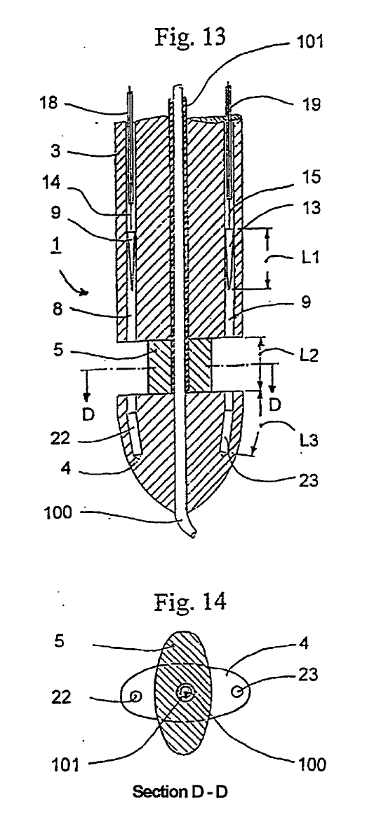

FIG. 13 shows an arrangement according to a further embodiment of the invention.

FIG. 14 shows a sectional view of the arrangement illustrated in FIG. 13 along the section line D-D indicated therein.

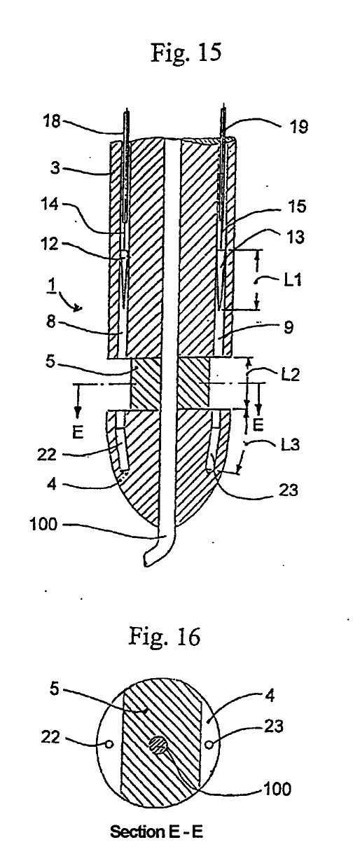

FIG. 15 shows yet a further embodiment of the arrangement according to the invention.

FIG. 16 shows a sectional view of the arrangement illustrated in FIG. 15 along the section line E-E indicated therein.

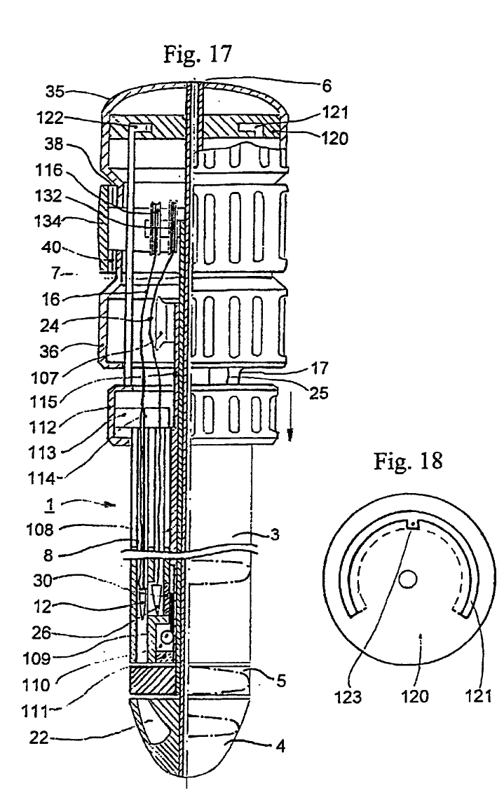

FIG. 17 shows a partially sectional view of yet a further embodiment of the arrangement according to the invention.

FIG. 18 shows a bottom view of a rotary plate belonging to a locking/release mechanism.

Before the specifics of the details illustrated in the drawings are discussed, it should first of all be pointed out, in general terms, that, within the context of the present application, in conjunction with the feed and accommodation of sutures, it is meant that the needles connected to the relevant sutures are also included in these operations.

FIG. 1 shows a largely rotationally symmetrical sectional view, in detail form, of a first embodiment of an arrangement according to the invention. The arrangement illustrated in FIG. 1 comprises a suture-guide device 1, which is illustrated in the bottom part of the relevant FIG. 1, and an operating or actuating device 2, which is illustrated in the top part of FIG. 1 and has a series of operating or actuating elements which will be explained in more detail hereinbelow. This arrangement is to be regarded as being illustrated on a vastly enlarged scale for the case where it serves for closing an opening provided in the wall of an artery of an individual, that is to say where it is used as a device for closing arteries. In the case of such an artery-closing arrangement, the dimension of the suture-guide device 1 in the cross-sectional direction is merely a few millimeters; a typical value, for example, is 4 mm.

According to FIG. 1, the suture-guide device 1 contains, as seen in the longitudinal direction of the relevant suture-guide device, a rear suture-feed part 3, a front suture-accommodating part 4 and a central suture-release/suture-clamping part 5 located therebetween. In the present case, these three arrangement parts 3, 4 and 5 are connected to one another in a rotatable manner via a double-tube or double-sleeve arrangement. The relevant sleeve arrangement contains an inner sleeve 6 and an outer sleeve 7 arranged coaxially therewith. According to FIG. 1, the inner sleeve 6 is fixed to the front suture-accommodating part 4, that is to say that provided at the distal end of the three arrangement parts 3, 4 and 5. The outer sleeve 7 is fixed to the central suture-release/suture-clamping part 5 and can be rotated relative to the inner sleeve 6 and also relative to the rear suture-feed part 3, that is to say that provided at the proximal end of the three arrangement parts 3, 4 and 5. This rotation is brought about by various elements of the abovementioned actuating device 2, which will be discussed in more detail hereinbelow.

In the case where the entire arrangement is an artery-closing arrangement, the central suture-release/suture-clamping part 5 has a thickness which corresponds to an artery-wall thickness. A typical value is approximately 2 mm. In this case, the length of the front needle-accommodating part 4 is typically approximately 6 mm.

The rear suture-feed part 3 has two longitudinal holes 8, 9 which, in the present case, are located at the edge of the suture-feed part 3 and are each covered here by a sheeting part 10 and 11, respectively, which may be adhesively bonded, for example, to the outer edge of the suture-feed part 3. It is thus possible for the relevant longitudinal holes 8, 9 to be positioned as far as possible in the outward direction, to be precise preferably to such an extent that part of the outer circumference of needles 12, 13 accommodated in said longitudinal holes 8, 9 is located outside the circumference of the rear suture-feed part 3. This is quite particularly beneficial for the case where the abovementioned needles are to act as far away as possible from the center of the suture-guide device 1, which will become clearer hereinbelow.

Connected to the abovementioned needles 12, 13 are sutures 14, 15 which each come from a suture supply contained in an associated suture magazine 16 and 17, respectively. The abovementioned sutures 14, 15 are guided in the abovementioned longitudinal holes 8, 9 by pushers 18, 19 which can be forced by means of an actuating key 43, counter to the spring force of springs 20, 21, in the direction of the front suture-accommodating part 4. The relevant pushers 18, 19 are formed here by tubular pushers.

Located in the front suture-accommodating part 4 are accommodating holes 22, 23 for accommodating the abovementioned needles 12, 13. In the present case, the accommodating holes 22, 23 in the front suture-accommodating part 4 have their respective inlet opening aligned in relation to the longitudinal holes 8 and 9, respectively, provided in the rear suture-feed part 3. According to FIG. 1, the accommodating holes 22, 23 have their respective longitudinal axis angled in relation to the longitudinal axis of the longitudinal holes 8, 9 in adaptation to the outer shape of the suture-accommodating part 4. It should also be pointed out here that the accommodating openings 22, 23 each have such a length that they are capable of accommodating in each case two needles, corresponding to the needles 12 and 13 respectively, one behind the other. This means that the accommodating openings 22, 23 each have a length which is at least twice the size of the length of each of the needles 12 and 13, respectively. In addition, the accommodating openings 22, 23 may be configured such that the needles 12 and 13, respectively, introduced into them in each case cannot readily be drawn out again. For this purpose, anchoring mechanisms known per se may be provided in the accommodating openings 22, 23.

Before the construction of the arrangement illustrated in FIG. 1 is discussed further, first of all the cross sections and/or shapes of the rear suture-feed part 3, of the front suture-accommodating part 4 and of the central suture-release/suture-clamping part 5 should be considered. The cross sections and/or shapes of these arrangement parts 3, 4 and 5 are illustrated in FIGS. 2, 3 and 4 in accordance with the section lines A-A and B-B and C-C, respectively, depicted in FIG. 1. Accordingly, the rear suture-feed part 3, the front suture-accommodating part 4 and the central suture-release/suture-clamping part 5 each have an oval-shaped cross section and/or an oval shape, in the present case the cross section and/or the shape of the central suture-release/suture-clamping part 5 having a smaller width than the cross sections and/or shapes of the rear suture-feed part 3 and of the front suture-accommodating part 4, as can be seen clearly by comparing FIG. 3 with FIGS. 2 and 4. In principle, therefore, all the arrangement parts 3, 4 and 5 each have an oval-shaped or elliptical cross section and/or a corresponding shape, in any case at least in the respectively adjacent regions. The longitudinal holes 8, 9 and the accommodating holes 22, 23 are located here in each case at diametrically opposite locations of the suture-feed part 3 and of the suture-accommodating part 4, respectively, to be precise on the longitudinal axis of the respective shaped body. In the case where the arrangement illustrated in FIG. 1 is used for closing an artery-wall opening, the suture-release/suture-clamping part 5 acts as a wound-edge tensioner which tensions the edge of the artery-wall opening, which constitutes a wound, such that, in the transverse direction of this tensioning, the needles 12, 13, and/or the needles 26, 27 which are yet to be considered, with their sutures can be guided through the artery wall.

FIGS. 5 and 6 show plan views of the central suture-release/suture-clamping part 5 and the front suture-accommodating part 4 in two different relative rotary positions. In the rotary position according to FIG. 5, which may be regarded as the starting position of the arrangement, the accommodating openings 22 and 23 contained in the front suture-accommodating part 4 are covered by the central suture-release/suture-clamping part 5. The left-hand suture-feed part 3 is located here in the same rotary position as the front suture-accommodating part 4. In the rotary position illustrated in FIG. 6, the relevant accommodating openings 22, 23 are freely accessible. As far as the arrangement illustrated in FIG. 1 is concerned, this means that, in the case of rotation of the central suture-release/suture-clamping part 5 relative to the front suture-accommodating part 4—this relative position is illustrated in FIG. 6—the needles 12 and 13 contained in the longitudinal holes 8, 9 can be guided in the direction of the accommodating openings 22 and 23, respectively, since their movement is released, rather than obstructed, in this position by the central suture-release/suture-clamping part 5.

Coming back to the arrangement illustrated in FIG. 1, it should be pointed out that in the rear suture-feed part 3, in addition to the already considered needles 12 and 13 with associated sutures, said arrangement also has additional needles 26 and 27 which are connected to further sutures 24 and 25, respectively, and are respectively provided in supply chambers 28 and 29 alongside the abovementioned longitudinal holes 8 and 9, respectively. The relevant supply chambers 28, 29 contain pressure-exerting plates 30 and 31, respectively, which are subjected to loading by compression springs and by means of which the additional needles 26 and 27, respectively, are forced in the direction of the abovementioned longitudinal holes 8 and 9, respectively. In that position of the arrangement which is illustrated in FIG. 1, this exertion of pressure as yet has no further effect; the addition needles 26 and 27 remain in the supply chambers 28 and 29, respectively. It is only when the pushers 18 and 19, once the needles 12 and 13 initially provided in the longitudinal holes 8 and 9, respectively, have been introduced into the accommodating openings 22 and 23, respectively, are drawn back to such an extent that the abovementioned supply chambers 28 and 29, respectively, are exposed that the abovementioned exertion of pressure results in the additional needles 26 and 27 then passing into the abovementioned longitudinal holes 8 and 9, respectively, in order then to be pushed into the respective accommodating openings 22 and 23 by the abovementioned pushers 18 and 19, respectively.

The further sutures 24 and 25 connected to the abovementioned additional needles 26 and 27, respectively, belong to suture supplies which are accommodated in separate suture magazines 32 and 33, respectively, in the rear suture-feed part 3.

The operating and/or actuating device 2 which belongs to the arrangement and is illustrated in FIG. 1 will now be considered in more detail. This actuating device 2 contains a hand grip 34 which is fixed to the abovementioned outer sleeve 7 of the arrangement. The entire arrangement according to FIG. 1 can be held using this hand grip. The actuating device 2 also contains a rotary adjustment wheel 35, which is fixed to the abovementioned inner sleeve 6, and a further rotary adjustment wheel 36, which is fixed to the rear suture-feed part 3. On its side which is located at the top in FIG. 1, the hand grip 34 has, at certain locations, latching holes 37 in which a latching catch 38, connected to the rotary adjustment wheel 35, is capable of engaging. This latching catch 38, which is merely schematically indicated by a spring-loaded rod arrangement, can be raised out of its respective latching position by means of an actuating lever 39, with the result that, following this raising-out operation, a relative rotation between the rotary adjustment wheel 35 and the hand grip 34 is possible.

On its side which is located at the bottom in FIG. 1, the hand grip 34 likewise has one or more latching holes 42 in which a latching catch 40, likewise merely schematically indicated as a spring-loaded rod arrangement, is capable of engaging. Actuation of an actuating lever 41 allows the latching catch 40 to be raised out of the respective latching opening 42 of the hand grip, with the result that relative rotation between the rear suture-feed part 3 and the hand grip 34 is then made possible.

As will become clear hereinbelow, the latching openings 37 and 42 in the hand grip 34 are provided in each case at quite specific locations, which allow a quite specific discharge of the needles 12, 13 and 26, 27 contained in the rear suture-feed part 3, as will be explained hereinbelow with reference to FIGS. 7 to 9.

FIG. 7 shows the relative rotation of the front suture-accommodating part 4 in relation to the central suture-release/suture-clamping part 5 in a first operation position, in which the accommodating openings 22, 23 are located on a straight line which encloses an angle of 45° in relation to the longitudinal axis running in the longitudinal direction of the suture-release/suture-clamping part 5. It should be pointed out here that the rear suture-feed part 3 of the arrangement is located congruently with the front suture-accommodating part 4. This means that, in this position or piercing plane, the longitudinal holes 8, 9 of the rear suture-feed part are aligned in relation to the accommodating openings 22, 23 of the front suture-accommodating part 4.

FIG. 8 shows the relevant arrangement in a second operating position or piercing plane, in which the front suture-accommodating part 4 has been rotated further through 90° in relation to the central suture-release/suture-clamping part 5. It goes without saying that, in this second operating position, it is also the case that the rear suture-feed part 3, which is not illustrated in FIG. 8, is located in the same position in relation to the central suture-release/suture-clamping part 5 as the front suture-accommodating part 4.

Whereas the needles 12 and 13 with their sutures 14 and 15, illustrated in FIG. 1, are accommodated by the accommodating openings 22 and 23, respectively, in the first operating position of the individual arrangement parts, said first position being illustrated in FIG. 7, the additional needles 26 and 27 with their sutures 24 and 25, respectively, are introduced into the accommodating openings 22 and 23, respectively, in the second operating position, which is illustrated in FIG. 8. It is thus possible, as is illustrated in FIG. 9, for four suturing holes 51, 52, 53, 54 to be pierced by the relevant needles in a wall 50 of a membrane, of a balloon or of a surface, which may be in particular an artery of an individual, in which there is located an opening which has its cut surface positioned against the outer circumference of the central suture-release/suture-clamping part 5. These needles are introduced, together with their sutures, into the accommodating openings 22 and 23 of the front suture-accommodating part 4. The suturing holes 51, 52 and 53, 54 are located on a straight line in each case, the resulting straight lines intersecting one another, for example, at an angle of 90°.

Latching holes which correspond to the rotary positions explained above with reference to FIGS. 7 to 9 and to further determined adjustment positions (e.g. rest or starting position) of the front suture-accommodating part 4 and of the rear suture-feed part 3 relative to the central suture-release/suture-clamping part 5 are provided in the hand grip 34 illustrated in FIG. 1. In conjunction with the abovementioned latching catches 38 and 40, it is thus possible for the adjustment wheels 35 and 36 connected to the arrangement parts 3 and 4 to be easily moved into the respectively desired rotary positions.

FIGS. 10 and 11 illustrate an added development of the above-explained arrangement according to the invention. According to this added development, on its surface directed towards the central suture-release/suture-clamping part 5, the front suture-accommodating part 4 has swing-open and swing-in spring elements 60, of which in the present case—as can be seen clearly from FIG. 11—two are provided. The two spring elements 60 each have a fastening surface 61, which is attached to the front suture-accommodating part 4, and swing-out and swing-in surfaces 62. The relevant spring elements 60 are configured here such that, with rotation of the front suture-accommodating part 4 from its starting position (as is illustrated, for example, in FIG. 5) relative to the central suture-release/suture-clamping part 5, they swing open in a first direction and thus increase the size of the surface of the front suture-accommodating part 4 in the direction of the central suture-release/suture-clamping part. With rotation of the front suture-accommodating part 4 back into its abovementioned starting position, the relevant spring elements 60 can have their surfaces 62 moved back into the swung-in state again. This measure of providing additional spring elements 60 may thus increase the size of the surface by which the front suture-accommodating part 4 butts against a wall through which the relevant front suture-accommodating part 4 is inserted. This allows, if appropriate, reliable functioning of the arrangement according to the invention considered.

FIG. 12 illustrates a modification, in detail form, of the arrangement according to the invention illustrated in FIG. 1. According to FIG. 12, additional needles 80, 81 are accommodated in supply chambers 82 and 83, respectively, formed alongside the longitudinal holes 8 and 9, respectively, on the outside of the overall arrangement. With the aid of pressure-exerting plates 84 and 85, which are connected to helical springs, the relevant needles 80 and 81, respectively, are forced in the direction of the abovementioned longitudinal holes 8 and 9, respectively. Sutures 86 and 87 running on the outside of the arrangement are connected here to the abovementioned needles 80 and 81, respectively.

Provided in the front suture-accommodating part 4 according to FIG. 12, in contrast to the conditions illustrated in FIG. 1, are two accommodating openings 88, 89 which are each respectively capable of accommodating two needles 12, 80 and 13, 81 one beside the other. In order for the needles 12 and 13 introduced first of all into the accommodating openings 88, 89 to be moved out of the introduction path for the additional second needles 80 and 81, respectively, FIG. 12 provides magnets 90 and 91 in the accommodating openings 88 and 89, respectively, for the case where at least the needles 12, 13 are magnetically attractable needles. These magnets attract the needles 12 and 13 introduced into the relevant accommodating openings and secure them.

The rest of the construction and configuration of the arrangement illustrated in FIG. 12 corresponds to the arrangement explained with reference to FIG. 1.

FIGS. 13 and 14 illustrate a further embodiment of the arrangement according to the present invention. This embodiment differs from the embodiment considered above in that the arrangement contains merely two needles 12, 13, to which sutures 14 and 15, respectively, are connected. In contrast to the embodiment considered according to FIG. 1 or 12, it is nevertheless the case, in the case of the embodiment according to FIGS. 13 and 14, that the cross section of the rear suture-feed part 3, of the front suture-accommodating part 4 and the central suture-release/suture-clamping part 5 is of oval-shaped design in each case, this oval-shaped cross section, more specifically, being provided at least in the adjacent regions of the respectively adjacent parts.

In contrast to the conditions illustrated in more detail in FIGS. 1 and 12, the embodiment according to FIGS. 13 and 14 provides a guide element which is formed by a guide wire 100, is connected to the front suture-accommodating part 4 and allows the latter to be rotated relative to the rear suture-feed part 3 and the central suture-release/suture-clamping part 5. The abovementioned central suture-release/suture-clamping part 5 is fixed to a sleeve part 101 which can be rotated relative to the guide element 100 and the rear suture-feed part 3.

In conjunction with the arrangement according to the invention illustrated in FIGS. 13 and 14, it should also be pointed out that the length L3 of the accommodating openings 22 and 23 which is depicted in FIG. 13 is at least equal to the dimension Li, that is to say the length of one of the needles 12, 13. The dimension L2 corresponds at least to the thickness of the wall through whose opening the arrangement illustrated is guided, in order for it to be possible for the needles 12 and 13 with the sutures 14 and 15, respectively, connected thereto to be guided through the relevant wall in the vicinity of the edge region of said opening.

FIGS. 15 and 16 illustrate yet a further embodiment of the arrangement according to the present invention. This most straightforward embodiment of the embodiments of the arrangement according to the invention which are explained here illustrates that the present invention also functions, in principle, when the rear suture-feed part 3 and the front suture-accommodating part 4 each have a circular cross section and when the central suture-release/suture-clamping part 5 has a smaller cross section in comparison. It may be sufficient here for said central suture-release/suture-clamping part 5 to be fixed to the rear suture-feed part 3, as a part of the latter, or even to be formed together therewith.

FIGS. 15 and 16 illustrate, as in FIGS. 13 and 14, a guide wire passing through all the arrangement parts, with the aid of which the front suture-accommodating part 4 can be rotated relative to the central suture-release/suture-clamping part 5, and thus relative to the rear suture-feed part 3. In the present case, this rotatability also realizes the same functions as have been mentioned above in conjunction with FIGS. 5 and 6.

As far as the lengths L1, L2 and L3 are concerned, what has been said in relation to FIGS. 13 and 14 applies here.

To conclude, it should also be pointed out that it is also possible for the guide elements in the form of guide wires which are mentioned in conjunction with the arrangements according to FIGS. 13 to 16 to be provided in the embodiments illustrated in FIGS. 1 to 12, although in the latter case merely as straightforward guide elements with no associated rotary function as has been explained in conjunction with FIGS. 13 to 16.

FIG. 17 illustrates yet a further embodiment of the arrangement according to the invention. Largely a half-section has been selected for the relevant illustration here, although a full section is provided in the top part of FIG. 17. The relevant illustration here is on a vastly enlarged scale, to be precise in the bottom part in particular, in order to illustrate details which will be discussed more specifically hereinbelow. Since the half-section illustration only illustrates the specifics of the construction in one half of the relevant arrangement, it is also just this construction which will be described in more detail hereinbelow. It goes without saying that, in its other half which is not illustrated in section, the arrangement is constructed at least largely correspondingly. In addition, it should also be pointed out that it is basically the case in FIG. 17 that those elements which correspond to the elements which have a comparable or the same function and are illustrated in FIG. 1 have the same designations as in FIG. 1.

In the same way as the arrangement illustrated in FIG. 1, the arrangement 1 illustrated in FIG. 17 has, in its suture-guide device 1, a suture-feed part 3, a suture-release/suture-clamping part 5 and a suture-accommodating part 4 in the relevant sequence from the proximal end to the distal end of the arrangement illustrated. The relevant parts 3, 4, 5 here each have an oval-shaped or elliptical cross section, as is indicated by chain-dotted lines in FIG. 17. Located in the suture-feed part 3 are longitudinal holes 8, 108 for accommodating the sutures 16 and 24 connected to the needles 12 and 26, respectively, as well as corresponding longitudinal holes for accommodating the sutures 17, 25 connected to needles which cannot be seen. In this case, the needle 26 is accommodated in an accommodating chamber in the bottom region of the suture-feed part 3 in a manner corresponding to that which has already been shown in FIG. 1. Accordingly, by means of a pressure-exerting plate 30, the needle 26 is subjected to pressure directed towards the left in FIG. 17. This pressure comes from a spring 109 which is accommodated in an accommodating chamber 110 which is formed in the bottom part of the suture-feed part 3 and, according to FIG. 17, is closed in the direction of the suture-release/suture-clamping part 5 by a closure part 111.

According to FIG. 17, the suture-accommodating part 4 likewise has accommodating holes, of which merely the accommodating hole 22 is illustrated specifically. This accommodating hole 22 and its corresponding accommodating hole in the other region of the arrangement illustrated are designed in fish-trap form in the present case. This has the advantage that a needle, once introduced into the relevant accommodating hole, cannot readily be drawn out of said accommodating opening again since—as is usually the case—it is no longer located in this accommodating hole in the position in which it was introduced. This applies, in particular, to the case where the suture connected to the relevant needle is connected laterally to said needle in relation to the longitudinal direction of the same.

Provided at the top, that is to say in this case proximal, end of the arrangement section which comprises the parts 3, 4, 5 is a displacement part 112 which, on its inner circumference, has a pushing element 113 formed by a flat plate. Fixed to said pushing element 113 is a pusher which is formed, in particular, by a spring wire 114 and with the aid of which the needle 12 or 26 located in the longitudinal hole 8 in each case can be displaced in the longitudinal direction, that is to say downwards in FIG. 17. For this purpose, the displacement part 112 can be displaced in the longitudinal direction of the suture-feed part 3, that is to say downwards according to FIG. 17. For this purpose, the longitudinal holes 8 and 108 accommodating the sutures 16, 24 are slotted such that they are capable of accommodating the relevant pushing element 113. It should be pointed out here that, in practice, the arrangement is such that the relevant pushing element 113 enters into slots of the material parts bounding the longitudinal holes 8, 108 at right angles to the position illustrated in FIG. 17. The respective slot depth is selected here so as to ensure that the respective needle 12, 26 can pass into the accommodating hole 22.

The displacement part 112 considered above is indeed, as has been described, longitudinally displaceable in the direction of the distal end of the arrangement illustrated; in the direction of rotation, however, it is fixed to the rotary adjustment wheel 36 directly adjacent to it. For this purpose, an outer sleeve 115, which is accommodated by a fastening part 107 in the rotary adjustment wheel 36, is provided according to FIG. 17, as it were, as part of the extension of the suture-feed part 3.

Provided above the adjustment wheel 36 considered above, according to FIG. 17, is an adjustment wheel 134 that corresponds to the hand grip 34 in FIG. 1 and is fixed to a sleeve 7, although, in contrast to the conditions illustrated in FIG. 1, it is a central sleeve here. In the present case, suture reels 116, 132, which correspond to the suture magazines 16, 32 illustrated in FIG. 1, are arranged in a rotatable manner in the rotary adjustment wheel 134, it being possible for the abovementioned sutures 16 and 24, respectively, to be drawn off from said reels. In the present case, the suture-release/suture-clamping part 5 can be rotated in relation to the suture-feed part 3 and the suture-accommodating part 4 by means of the rotary adjustment wheel 134, which corresponds to the conditions which have already been explained in conjunction with FIG. 1.

The rotary adjustment wheel 35, which is also provided in FIG. 1, is shown in the top part of FIG. 17 and is connected to the inner sleeve 6, via which the suture-accommodating part 4 can be rotated relative to the suture-feed part 3 and the suture-release/suture-clamping part 5. The inner sleeve 6 in this case, as is also illustrated in FIG. 1, is a hollow sleeve through which a guide wire, such as the guide wire 100, can be guided as guide element.

In order for it to be possible for the rotary adjustment wheels 35, 134, 36 illustrated in FIG. 17 to be adjusted relative to one another with latching action into respectively desired positions, it is the case, as with the embodiment illustrated in FIG. 1, that latching catches 38 and 40 are provided between the rotary adjustment wheel 134 and the two adjacent rotary adjustment wheels 35 and 36. According to FIG. 17, however, these latching catches, rather than being provided in the longitudinal direction of the arrangement illustrated, are provided in the radial direction between the relevant rotary adjustment wheels.

In addition to the elements considered above, by way of which the arrangement illustrated in FIG. 17 corresponds in principle to the arrangement illustrated in FIG. 1, FIG. 17 also provides a separate locking/release mechanism, with the aid of which it is ensured that the rotary adjustment wheels 35 and 36 for the suture-feed part 3 and for the suture-accommodating part 4 only release a displacement of the needles by the respective pushers when the suture-feed part 3 and the suture-accommodating part 4 are aligned in relation to one another. In the present case, this locking/release mechanism contains a rotary plate 120 which is connected to the rotary adjustment wheel 35 and has an L-shaped cutout 121 in which there is accommodated a locking rod 122 which is fixed to the displacement part 112. FIG. 18 illustrates the relevant rotary plate 120 as seen from beneath. It can-be seen here that this rotary plate 120 has a recess 123 at a certain position, in the present case in its central position, in the region of the L-shaped cutout 121 which runs concentrically with the outside of the relevant rotary plate, it being possible, in the region of said recess, for the abovementioned locking rod 122 to be guided out of its locking position, that is to say released. It is only in this position that the displacement part 112 according to FIG. 17 can be displaced downwards. As has already been mentioned above, this position is that position in which the suture-feed part 3 and the suture-accommodating part 4 are aligned in relation to one another and in which, moreover, the suture-release/suture-clamping part 5 is also preferably in its suture-release position.

The locking/release mechanism considered above has been explained in conjunction with the insertion of the needles in a single piercing plane. This piercing plane and/or its position is defined here by the determination of the recess 123. It should be possible to appreciate, however, that in the case where a plurality of piercing planes are provided, as has been explained, for example, with reference to FIGS. 7 to 9, a number of recesses, corresponding to the recess 123, which corresponds to this number of piercing planes will be provided at such locations of the L-shaped cutout 121 as correspond to the abovementioned piercing planes and/or the positions thereof. It is also possible here for the angles between the individual piercing planes to have values other than those which have been explained in conjunction with FIGS. 7 to 9, for example a value of 60° in each case.

As an addition to the arrangement 1 illustrated in FIG. 17, it should also be pointed out that, in the case of this arrangement, the longitudinally running outsides of the rotary adjustment wheels 35, 36 and 134 more or less have a common outer line, whereas the displacement part 112 has its outside offset inwards in comparison. In contrast to this, however, it is also possible to use other configurations.

In the region between the displacement part 112 and the rotary adjustment wheel 36 directly adjacent thereto, the sutures 17, 25 are also indicated in FIG. 17 in addition to the sutures 16, 24 and the locking rod 122. In this case, the region between the displacement part 112 and the rotary adjustment wheel 36 is illustrated as an open region. In practice, however, this region may be closed in the outward direction by corresponding screening parts which are connected, for example, telescopically to the rotary adjustment wheel 36 and the displacement part 112.

In addition, it should also be pointed out that, rather than the invention being restricted to the embodiments described, it is possible to provide in the rear suture-feed part 3 and in the front suture-accommodating part 4 in each case m groups, where m≧2, of n longitudinal and accommodating holes located one beside the other, where n≧1. Where m≧2, this results in geometrical arrangements for the individual arrangement parts which differ from the geometrical arrangements illustrated in the drawings. Where m>3, for example, this results in a triangular cross-sectional configuration for the arrangement according to the invention.

Arrangement for guiding at least two sutures through a wall, in particular of an artery of an individual, in the vicinity of the edge region of an opening provided therein

Addition to (DE 199 42 951.0-C1)

The main patent (DE 199 42 951.0-C1) relates to an arrangement for guiding at least two sutures through a wall of a membrane, of a balloon or of a surface, in particular of an artery of an individual, in the vicinity of the edge region of an opening provided therein, and formed if appropriate by cutting in and/or cutting out, and for drawing back out of the abovementioned opening the suture ends guided through the relevant wall, having a shaft-like suture-guide device in which the sutures fastened on needles are guided in guide and/or accommodating openings such that, by way of the relevant suture-guide device, they can be guided through the wall of the relevant membrane or of the relevant balloon or blood vessel and drawn back out of the abovementioned opening again such that, by virtue of the suture ends being drawn together outside the relevant opening, the latter can be closed, it being the case that the suture-guide device, in its longitudinal direction, contains a rear suture-feed part, a front suture-accommodating part and a central suture-release/suture-clamping part located therebetween, and it being the case that the abovementioned central suture-release/suture-clamping part can be rotated at least relative to the front suture-accommodating part and has such a cross section that, in at least one rotary position, it allows the sutures fed from the rear suture-feed part to be introduced into accommodating openings exposed in the front suture-accommodating part and, in a rotary position differing from the abovementioned rotary position, it allows the sutures accommodated in the relevant accommodating openings together with the needles to be secured for drawing the entire suture-guide device out of the abovementioned openings.

Although the relevant arrangement works satisfactorily, it is occasionally desired to increase further the tensioning of the abovementioned wall of a membrane, of a balloon or of a surface, in particular of an artery of an individual, in the vicinity of the edge region of an opening provided therein, and formed if appropriate by cutting in and/or cutting out, said tensioning being achieved by the central suture-release/suture-clamping part used. This applies, in particular, to the case where the relevant wall is formed by an artery of an individual in which the abovementioned central suture-release/suture-clamping part of the relevant arrangement acts, as it were, as a wound-edge tensioner.

Accordingly, the object of the invention is to find a way in which, in the case of an arrangement of the type mentioned in the introduction, the needles which are to be guided together with sutures through the wall of the abovementioned membrane, of the abovementioned balloon or of the abovementioned surface, in particular of an artery of an individual, can be guided through in a particularly effective manner at a reliable distance from the respective wound edge such that the respective wall does not tear.

The above object is achieved according to the invention, in the case of an arrangement of the type mentioned in the introduction, in that the abovementioned central suture-release/suture-clamping part can be expanded in one axial direction such that that wall of the membrane, of the balloon or of a surface, in particular of a vessel wall of an artery of an individual, which is located in the region of the relevant central suture-release/suture-clamping part is thus subjected to tensioning, on account of which those edge regions of the relevant membrane, balloon or vessel wall which are provided transversely to said tensioning direction draw together toward one another.

By virtue of those edge regions of the relevant membrane, balloon or surface or vessel wall having drawn together in the abovementioned direction, on account of the abovementioned tensioning, the respective membrane, balloon or surface or artery edge region butts particularly reliably, and thus firmly, against the outer circumference or edge region of the suture-release/suture-clamping part, with the result that the needles with their sutures can be guided through the relevant wall edge regions there at a reliable distance without the relevant edge regions tearing. This is advantageous, in particular, in cases of small to very small openings in a vessel wall, in the case of which the locations where the abovementioned needles pierce the relevant vessel-wall regions are very small, for example of the order of magnitude of from 0.5 to 1.5 mm. It is also advantageous for it to be possible for the working diameter of the arrangement according to the main patent and containing the central suture-release/suture-clamping part and the suture-feed part and the suture-accommodating part to be selected to be smaller than hitherto. Expansion of the relevant central suture-release/suture-clamping part in accordance with the respective application case thus makes available a universally useable arrangement for closing openings of different sizes in a wall of a membrane, of a balloon or of a surface, in particular of an artery of an individual.

The central suture-release/suture-clamping part can expediently be expanded mechanically and/or electrically and/or pneumatically/hydraulically. This allows the relevant central suture-release/suture-clamping part advantageously to be expanded in accordance with a wide range of different wishes and/or requirements.

It is also advantageous for the central suture-release/suture-clamping part to be expanded in the abovementioned one axial direction in that at least one edge part of the relevant suture-release/suture-clamping part which is located in said axial direction can be moved in the outward direction. This advantageously allows a particularly straightforward design of the central suture-release/suture-clamping part for movement in the outward direction.

It is also advantageous for the central suture-release/suture-clamping part to be divided up into a central part and into two adjacent edge parts which can be moved relative to the relevant central part. This makes it possible to realize, in a particularly straightforward manner, a symmetrically arranged suture-release/suture-clamping part for the edge regions of the latter to be moved in the outward direction.

The respective edge part of the central suture-release/suture-clamping part is expediently connected to a pushing and pulling device, the actuation of which can displace the respective edge part relative to the central part. This allows the respective edge part to be moved in the outward direction in a particularly straightforward manner.

It is also possible, however, for the central suture-release/suture-clamping part to have two clamping parts which, in an inactive state, are accommodated by an accommodating tube and which, in the latter, are subjected to an outwardly directed movement pressure by a force-exerting device, in particular a spring, and which, once the relevant accommodating tube has been drawn back, can be forced away from one another into an active state. This measure has the advantage that it is possible to manage without a separate pushing device in order to move the abovementioned two clamping parts in the outward direction for expansion of the central suture-release/suture-clamping part.

The two clamping parts which have just been mentioned can expediently be guided toward one another by means of a pulling device. This allows the two abovementioned clamping parts to be moved back into the inactive state again in a particularly straightforward manner.

According to a further expedient configuration of the invention, the abovementioned edge parts are connected to the relevant central part via pneumatic/hydraulic chambers and can be moved relative to the abovementioned central part by pneumatic/hydraulic action. This gives the advantage of a particularly straightforward design for a movement of the abovementioned edge parts relative to the abovementioned central part.

According to a further expedient configuration of the invention, the abovementioned edge parts can be moved relative to one another by means of a slotted-guide control device. This results in a particularly stable design in order for it to be possible for the abovementioned edge part to be displaced relative to the abovementioned central part.

It has turned out to be particularly advantageous if, in its outer region, the central suture-release/suture-clamping part is designed concavely for abutment against the edge region of the abovementioned membrane, balloon or surface, in particular vessel, wall. As a result, the respective membrane, balloon or surface or vessel wall butts reliably against the outer circumference region of the suture-release/suture-clamping part and cannot readily move out of said abutment region.

The invention is explained in more detail hereinbelow, by way of example, with reference to drawings.

FIG. 1 shows, of the arrangement described and illustrated in the main patent, merely a suture-feed part, a suture-accommodating part and a suture-release/suture-clamping part located therebetween, in a schematic view.

FIG. 2 shows a sectional view of the suture-release/suture-clamping part indicated schematically in FIG. 1 along the section plane X-X indicated in FIG. 1.

FIG. 3 shows a sectional view of another embodiment of the relevant suture-release/suture-clamping part, in an inactive position.

FIG. 4 shows the arrangement illustrated in FIG. 3 in an active state.

FIGS. 5 and 6 show a schematic illustration of yet a further embodiment of the abovementioned suture-release/suture-clamping part.

FIG. 7 shows a further schematic illustration of yet a further alternative embodiment of the relevant suture-release/suture-clamping part.

Before the details illustrated schematically in the drawings are discussed, it should be pointed out in principle beforehand that it is only those arrangement parts by which the present invention differs from the arrangements included in the main patent which are explained here.

FIG. 1 indicates schematically a suture-feed part 3, a suture-accommodating part 4 and a central suture-release/suture-clamping part 5, as are provided in the various arrangements included in the main patent. According to FIG. 1, the central suture-release/suture-clamping part 5 is located in the suture-release position, in which needles with sutures can be moved through the indicated openings 8, 22 and 9, 23 of the suture-feed part 3 and the suture-accommodating part 4 and can thus be guided through the edge region of an opening in a membrane, balloon or surface part, in particular of an artery of an individual, located between said parts 3 and 4. The relevant membrane, balloon or surface or artery edge region here butts against the outer circumference of the suture-release/suture-clamping part 5 such that there is a distance D1, D2 between the respective edge region and the center of the respective needle. It should also be pointed out here that the suture-feed part 3, the suture-accommodating part 4 and the central suture-release/suture-clamping part 5 are indicated in each case as parts having an oval-shaped cross section; however, the invention is not restricted to this. It is thus possible for the relevant parts each to have, for example, a circular cross section; the abovementioned suture-release/suture-clamping part 5 may additionally have an elongate cross-sectional shape, as is shown in the main patent.

In order to allow the respective membrane, balloon or surface or artery edge region to butt particularly reliably against the outer circumference or edge region of the suture-release/suture-clamping part 5, the present invention makes provision for the relevant suture-release/suture-clamping part 5 to be expanded in one axial direction, which is in direction X-X according to FIG. 1. This axial direction runs from left to right according to FIG. 1 and thus at right angles to the axis Y-Y, on which the openings 8, 22 and 9, 23 are located. According to FIG. 1, this expansion of the suture-release/suture-clamping part 5 takes place symmetrically about the center axis of the relevant part 5, in the direction of the arrows A and B indicated there. In the case where the relevant arrangement is used for closing an opening provided in a vessel wall, such as an artery, this expansion of the suture-release/suture-clamping part 5 causes this part 5 to act for tensioning this opening, and thus as a wound-edge tensioner. That wall of the membrane, of the balloon or of a surface or of a vessel wall, in particular of an artery of an individual, which is located in the region of the relevant central suture-release/suture-clamping part 5 is thus subjected to tensioning, on account of which those edge regions of the relevant membrane, balloon or surface or vessel wall which are provided transversely to said tensioning direction (X-X), that is to say in the direction Y-Y, draw together toward one another. This means that the relevant membrane, balloon or surface or vessel wall is thus positioned very closely against that region of the suture-release/suture-clamping part 5 which runs in the transverse direction Y-Y in relation to the relevant tensioning direction X-X. It is thus ensured that the needles guided in the region of the abovementioned openings 8, 22 and 9, 23, rather than piercing any lacerated membrane, balloon or vessel-wall region, actually pierce a smooth edge, this avoiding any tearing of the respective edge region of the membrane, balloon or vessel wall in the region of the respective needle-insertion location. Incidentally, a corresponding effect is also achieved in the cases where the suture-release/suture-clamping part 5 is expanded along one or more axes which, although running transversely to the axis Y-Y, are each located at any angle other than 90° in relation to the same.

FIG. 1 shows, schematically, that the abovementioned expansion of the central suture-release/suture-clamping part 5 is possible in that this part 5 is divided up into a central part 5M and into two adjacent edge parts 5R1 and 5R2. The edge parts 5R1, 5R2 are connected to the central part 5M via guide elements F1, F2 and F3, F4, respectively. As can be gathered from the schematic sectional view illustrated in FIG. 2, these edge parts 5R1, 5R2 can be moved mechanically relative to the central part 5M by pushing and pulling wires DZ1, DZ2. It should be pointed out here that it is also possible, in principle, for the arrangement to be such that only one of the edge parts 5R1, 5R2 of the suture-release/suture-clamping part 5 which has just been mentioned can be moved outward in the abovementioned axial direction, that is to say in the direction X-X specified in FIG. 1.

Another alternative embodiment of the central suture-release/suture-clamping part according to the invention is indicated schematically in FIGS. 3 and 4. According thereto, the central suture-release/suture-clamping part 5 has two clamping parts K1, K2 which, in an inactive state, are accommodated by an accommodating tube R and, in the latter, are subjected to an outwardly directed movement pressure by a force-exerting device, in particular by a spring Fe1, Fe2, respectively, and which, once the relevant accommodating tube R has been drawn back, can be forced away from one another, in the direction of the arrows C and D, into an active state. The respective spring Fe1, Fe2 is preferably a compression spring here. The relevant conditions are illustrated clearly in FIGS. 3 and 4. In order for the abovementioned two clamping parts K1, K2 to be guided together again, as is illustrated in FIG. 3, there is preferably provided a pulling device in the form of pulling wires Z1, Z2, with the aid of which the clamping parts K1, K2 can be drawn together again, with the result that the abovementioned accommodating tube R can then be pushed over the clamping parts K1, K2 again.

A further modification of the central suture-release/suture-clamping part according to the invention is illustrated in FIGS. 5 and 6. FIGS. 5 and 6 both illustrate a slotted-guide control device comprising two rotary parts DT1, DT2, as illustrated in FIG. 5, and edge parts 5T1 and 5T2 which form the actual central suture-release/suture-clamping part 5 and in which a guide slot Ku1, Ku2 is respectively located. The rotary parts DT1, DT2 engage in said guide slots by way of corresponding studs Z1 and Z2, respectively. By virtue of rotation of the rotary parts DT according to FIG. 5, it is possible for the two edge parts 5T1 and 5T2 of the suture-release/suture-clamping part 5 illustrated in FIG. 6 to be moved away from one another or toward one another in the direction of their longitudinal axis, as is indicated by arrows E and F in FIG. 6.

FIG. 7 illustrates a further exemplary embodiment of the suture-release/suture-clamping device according to the invention. FIG. 7 here shows a suture-release/suture-clamping part 5 of which the edge parts RT1, RT2 are connected to a central part MT via pneumatic/hydraulic chambers Ka1 and Ka2, respectively, and can be moved relative to the abovementioned central part MT by pneumatic and/or hydraulic action, as is indicated in FIG. 7 by arrows G and H. The corresponding pressure medium or hydraulic medium may be directed here into the relevant chambers Ka1 and Ka2, for example, from a central opening of the central part MT, and guided out of the same, by way of small pipelines, as is indicated in FIG. 7.

In conjunction with the exemplary embodiments explained above in relation to the drawings, it should become clear that the central suture-release/suture-clamping part 5 can be expanded in each case mechanically or pneumatically/hydraulically. It should be possible to appreciate that these methods of mechanical or pneumatic/hydraulic expansion may also take place in combination with one another, and that it is also possible for electrical expansion to take place either alone or in addition to such expansion measures. This electrical expansion can take place, for example, with the aid of piezoelectric actuators which allow the respective edge parts to be displaced relative to the respective central part of the suture-release/suture-clamping part 5.

In addition, it should also be pointed out that, in its outer or edge region, the central suture-release/suture-clamping part 5 is preferably designed concavely for abutment against the edge region of the abovementioned membrane, balloon or surface or vessel wall, as is illustrated particularly clearly in FIGS. 2, 3 and 4. It goes without saying that such a concave recess Ak, of course, may also be useful in all the other exemplary embodiments according to the invention.

To conclude, it should also be pointed out that the expansion path of the respective suture-release/suture-clamping part 5 depends on the respective application of the arrangement according to the main patent which contains said suture-release/suture-clamping part. It is thus possible, for example, in a membrane or a balloon, for the abovementioned suture-release/suture-clamping part 5 to be expanded by, for example, 5 to 10 mm in the case of an opening which is to be closed having a hole size of, for example, 10 to 20 mm. In the case of the relevant arrangement used for closing an opening which is formed in an artery wall of an individual and is, for example, 3 to 5 mm, the abovementioned suture-release/suture-clamping part 5 may be expanded by, for example, 1 to 2.5 mm.

### Description

Arrangement for eliminating, or at least for reducing the size of, interspaces between three elements arranged one behind the other in the longitudinal direction

The invention relates to an arrangement for eliminating, or at least for reducing the size of, interspaces between a first and a second element and between the second element and a third element, all three elements being arranged one behind the other, along a common longitudinal axis, at the distal arrangement end and being rotatable relative to one another about the relevant longitudinal axis, the three elements being formed, in particular, by a suture-feed part, a suture-clamping part and a suture-accommodating part of an arrangement for guiding at least two sutures through a wall, in particular of an artery of an individual, in the vicinity of the edge region of an opening provided therein, it being the case that the first element, provided at the distal arrangement end, is connected, via an inner sleeve, to a first retaining or rotary adjustment part provided at the proximal arrangement end, it being the case that the second element, directly adjacent to the first element provided at the distal arrangement end, is connected to an outer sleeve, which encloses the abovementioned inner sleeve and is connected to a second retaining or rotary adjustment part, provided at a distance from the abovementioned first retaining or rotary adjustment part in the direction of the distal arrangement end, and it being the case that the third element, directly adjacent to the abovementioned second element at the distal arrangement end in the direction of the proximal arrangement end, is connected to a third retaining or rotary adjustment part, which is adjacent to the abovementioned second retaining or rotary adjustment part in the direction of the distal arrangement end.

The abovementioned arrangement for guiding at least two sutures through a wall, in particular an artery of an individual, in the vicinity of the edge region of an opening provided therein is specified in DE 199 42 951 C1. It may be the case with this arrangement that its abovementioned three elements or parts, namely the suture-feed part, the suture-clamping part and the suture-accommodating part are deflected in relation to one another during use of the relevant arrangement, with the result that an interspace or gap forms in each case between the adjacent parts, this interspace or gap making it difficult to work with the relevant arrangement.

The problem presented above, however, also arises in general in the case of an arrangement of the type mentioned in the introduction if three arrangement elements arranged one behind the other along a longitudinal axis can be deflected in relation to one another.

The object of the invention is thus to develop an arrangement of the type mentioned in the introduction such that, in a relatively straightforward manner, the abovementioned formation of an interspace or gap between adjacent elements can be avoided or at least kept within such narrow limits that no disruption is caused during use of the relevant arrangement.

The above object is achieved according to the invention, in the case of an arrangement of the type mentioned in the introduction, on the one hand in that in each case one spring device is provided between the first retaining or rotary adjustment part and the second retaining or rotary adjustment part and between the second retaining or rotary adjustment part and the third retaining or rotary adjustment part, said spring device forcing the respectively adjacent retaining or rotary adjustment parts away from one another. This has the advantage of a particularly low level of design outlay as far as eliminating, or at least reducing the size of, the formation of an interspace or gap between directly adjacent elements is concerned.

The above object is achieved according to the invention, in the case of an arrangement of the type mentioned in the introduction, on the other hand in that, with the second and third retaining or rotary adjustment parts coupled to one another in the direction of the abovementioned longitudinal axis, the outer sleeve, which is connected to the second retaining or rotary adjustment part, can be displaced relative to said second retaining or rotary adjustment part in the direction of the abovementioned longitudinal axis and at its proximal end, which is located between the first and second retaining or rotary adjustment parts, has an edge, in each case one spring device being provided between said edge and the respectively adjacent sides of the first and second retaining or rotary adjustment parts.

This has the advantage that, by a relatively straightforward measure, it is possible for the spring devices to be arranged merely in the region between the first and the second retaining or rotary parts in order to ensure the elimination, or at least the reduction in size of, the formation of an interspace or gap between directly adjacent elements. This design solution may be very considerably useful as far as the amount of space required is concerned.

The respective spring device is preferably formed by a compression spring. This has the advantage of a particularly low level of outlay for providing the respective spring device.

Two exemplary embodiments according to the invention are explained in more detail hereinbelow with reference to drawings.

FIG. 1 shows a sectional view of a first embodiment of the invention of an arrangement embodying the invention.

FIG. 2 shows a sectional view of a second embodiment of the invention of an arrangement embodying the invention.

FIG. 1 shows a largely rotationally symmetrical sectional view, in detail form, of a first embodiment of an arrangement 1 according to the invention. The relevant illustration here corresponds largely to the illustration shown in FIG. 1 of DE 199 42 951 C1. A more detailed description of this arrangement may thus be dispensed with here; it should be sufficient to discuss only those parts or elements of the relevant arrangement which are necessary for understanding the present invention.

The arrangement 1 illustrated in FIG. 1 has, in the longitudinal direction along a common axis of rotation, three elements arranged one behind the other at the distal arrangement end, to be precise a suture-accommodating part 4 as the first element, a suture-clamping part 5 as the second element, directly adjacent to the first element in the direction of the proximal arrangement end, and a suture-feed part 3 as the third element, directly adjacent to the second element in the direction of the proximal arrangement end.

The suture-accommodating part 4 is fixed to an inner sleeve 6, of which the proximal end is fixed to a rotary adjustment wheel 35. The suture-clamping part 5 is connected to the distal end of an outer sleeve 7, which encloses the inner sleeve 6 and is connected to a hand grip 34 at its proximal end. The outer sleeve 7, for its part, is enclosed by the suture-feed part 3, which is connected to a further rotary adjustment wheel 36 at its proximal end. The two rotary adjustment wheels 35, 36 and the hand grip 34 each constitute a retaining or rotary adjustment part.

In order, then, to avoid the formation of interspaces or gaps 11, 12 between the above-considered elements 4 and 5, on the one hand, and 5 and 3, on the other hand, or at least to keep the same within such narrow limits that they do not pose any problems during use of the relevant arrangement, the present invention provides, between the rotary adjustment wheels 35, 36 and the hand grip 34, spring devices 13, 14 which force the relevant rotary adjustment wheels 35, 36 away from the hand grip 34 and thus force the suture-accommodating part 4 and the suture-feed part 3 against the suture-clamping part 5. The relevant spring devices 13, 14 are preferably formed in each case by a helical compression spring which runs around the inner sleeve 6 and the outer sleeve 7, respectively.

FIG. 2 illustrates a second embodiment of the arrangement 1 according to the invention. This is based essentially on the same arrangement construction as is illustrated in FIG. 1. In contrast to the conditions given in FIG. 1, in the embodiment shown in FIG. 2 the hand grip 34 and the further rotary adjustment tool 36 are coupled firmly to one another in the direction of the longitudinal axis of the relevant arrangement by a rotationally symmetrical groove/tongue arrangement 15, 16, with the result that these elements 34 and 36 can be rotated relative to one another in the direction of rotation, but cannot be moved towards one another or away from one another in the longitudinal direction. In this case, however, the outer sleeve 7, which is connected to the hand grip 34, can be displaced relative to said hand grip 34 in the direction of the abovementioned longitudinal axis; in the direction of rotation, however, the hand grip 34 is coupled to the outer sleeve 7 such that no relative rotation is possible between these two elements.

At its proximal end, which according to FIG. 2 is located between the rotary adjustment wheel 35 and the hand grip 34, the abovementioned outer sleeve 7 has an edge 17, in each case one spring device 18, 19 being provided between said edge and the respectively adjacent sides of the rotary adjustment wheel 35 and the hand grip 34. These spring devices 18, 19 are likewise formed in each case by a helical compression spring, of which the spring device 18 forces the rotary adjustment wheel 35 away from the edge 17 of the outer sleeve 7 and thus forces the suture-accommodating part 4 against the suture-clamping part 5. The other spring device 19 likewise forces the hand grip 34, together with the further rotary adjustment wheel 36, away from the edge 17 of the outer sleeve 7, which results in the suture-feed part 3 being forced against the suture-clamping part 5. This achieves the same effect as far as eliminating, or at least reducing the size of, the interspaces or gaps 11, 12 between the suture-clamping part 5 and the suture-feed part 3 and between the suture-clamping part 5 and the suture-accommodating part 4 is concerned. In contrast to the embodiment shown in FIG. 1, the design shown in FIG. 2, however, has a construction in which the entire spring arrangement 18, 19, for eliminating, or at least for reducing the size of, the formation of interspaces or gaps, is accommodated merely in the region between the rotary adjustment wheel 35 and the hand grip 34, as a result of which the interspace between the hand grip 34 and the further rotary adjustment wheel 36 can be utilized for other design elements.

1. An arrangement for eliminating, or at least for reducing the size of, interspaces between a first and a second element and between the second element and a third element, all three elements being arranged one behind the other, along a common longitudinal axis, at the distal arrangement end and being rotatable relative to one another about the relevant longitudinal axis, the three elements being formed, in particular, by a suture-feed part, a suture-clamping part and a suture-accommodating part of an arrangement for guiding at least two sutures through a wall, in particular of an artery of an individual, in the vicinity of the edge region of an opening provided therein, it being the case that the first element, provided at the distal arrangement end, is connected, via an inner sleeve, to a first retaining or rotary adjustment part provided at the proximal arrangement end, it being the case that the second element, directly adjacent to the first element provided at the distal arrangement end, is connected to an outer sleeve, which encloses the abovementioned inner sleeve and is connected to a second retaining or rotary adjustment part, provided at a distance from the abovementioned first retaining or rotary adjustment part in the direction of the distal arrangement end, and it being the case that the third element, directly adjacent to the abovementioned second element at the distal arrangement end in the direction of the proximal arrangement end, is connected to a third retaining or rotary adjustment part, which is adjacent to the abovementioned second retaining or rotary adjustment part in the direction of the distal arrangement end, characterized in that in each case one spring device (13, 14) is provided between the first retaining or rotary adjustment part (35) and the second retaining or rotary adjustment part (34) and between the second retaining or rotary adjustment part (34) and the third retaining or rotary adjustment part (36), said spring device forcing the respectively adjacent retaining or rotary adjustment parts (35, 34; 34, 36) away from one another.

2. The arrangement for eliminating, or at least for reducing the size of, interspaces between a first and a second element and between the second element and a third element, all three elements being arranged one behind the other, along a common longitudinal axis, at the distal arrangement end and being rotatable relative to one another about the relevant longitudinal axis, the three elements being formed, in particular, by a suture-feed part, a suture-clamping part and a suture-accommodating part of an arrangement for guiding at least two sutures through a wall, in particular of an artery of an individual, in the vicinity of the edge region of an opening provided therein, it being the case that the first element, provided at the distal arrangement end, is connected, via an inner sleeve, to a first retaining or rotary adjustment part provided at the proximal arrangement end, it being the case that the second element, directly adjacent to the first element provided at the distal arrangement end, is connected to an outer sleeve, which encloses the abovementioned inner sleeve and is connected to a second retaining or rotary adjustment part, provided at a distance from the abovementioned first retaining or rotary adjustment part in the direction of the distal arrangement end, and it being the case that the third element, directly adjacent to the abovementioned second element at the distal arrangement end in the direction of the proximal arrangement end, is connected to a third retaining or rotary adjustment part, which is adjacent to the abovementioned second retaining or rotary adjustment part in the direction of the distal arrangement end, characterized in that, with the second and third retaining or rotary adjustment parts (34, 36) coupled to one another in the direction of the abovementioned longitudinal axis, the outer sleeve (7), which is connected to the second retaining or rotary adjustment part (34), can be displaced relative to said second retaining or rotary adjustment part (34) in the direction of the abovementioned longitudinal axis and at its proximal end, which is located between the first and second retaining or rotary adjustment parts (35, 34), has an edge (17), in each case one spring device (18, 19) being provided between said edge and the respectively adjacent sides of the first and second retaining or rotary adjustment parts (35, 34).

3. The arrangement as claimed in claim 1, characterized in that the respective spring device (13, 14, 18, 19) is formed by a compression spring.

4. The arrangement as claimed in claim 2, characterized in that the respective spring device (13, 14, 18, 19) is formed by a compression spring.
